(19) United States
(12) Patent Application Publication
(10) Pub. No.: US 2023/0155252 A1
(43) Pub. Date: May 18, 2023

(54) SURFACTANT COATED SEPARATOR

(71) Applicant: DARAMIC, LLC, Charlotte, NC (US)

(72) Inventors: Eric H. Miller, Philpot, KY (US); Margaret R. Roberts, Owensboro, KY (US); Naoto Miyake, Kanagawa-ken (JP); M. Neil Golovin, Silver Spring, MD (US)

(21) Appl. No.: 17/916,804

(22) PCT Filed: Apr. 5, 2021
(86) PCT No.: PCT/US2021/025796

§ 371 Date: Oct. 4, 2022

Related U.S. Application Data
(60) Provisional application No. 63/005,842, filed on Apr. 6, 2020.

(51) Int. Cl. H01M 50/489; H01M 50/417; H01M 10/06; H01M 50/449

(52) U.S. Cl. CPC H01M 50/489; H01M 10/06; H01M 50/417; H01M 50/449

(57) ABSTRACT

A battery separator including a porous membrane with a surfactant coating on at least one side thereof is described. The surfactant coating may include a non-ionic surfactant in a low amount, a blend of a non-ionic surfactant and an ionic surfactant, or the surfactant coating may contain only a compound having the following structures:

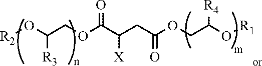

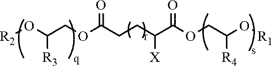

where n is an integer from 0 to 10, m is an integer from 0 to 10, n and m are the same or different, q is an integer from 0 to 10, r is an integer from 0 to 10, s is an integer from 0 to 10, and q, r, and s are the same or different, R1 is H, a C1 to C10 linear or branched, saturated or unsaturated alkyl group, a C1 to C10 fatty alcohol, a C1 to C10 alcohol, or an aromatic group, and R2 is H, a C1 to C10 linear or branched, saturated or unsaturated alkyl group, a C1 to C10 linear or branched, saturated or unsaturated fatty alcohol, a C1 to C10 linear or branched, saturated or unsaturated alcohol, or an aromatic group, n and m are the same or different, R1 and R2 are the same or different, R3 is hydrogen or methyl or a C1 to C5 alkyl group, R4 is hydrogen or methyl or a C1 to C5 alkyl group R3 and R4 are the same or different, and X is a negatively charged groups such as SO3—, COO—, PO4-2, and the like; and also a positively charged counterion. When the separator described herein is used in a battery, the battery may exhibit improved properties such as reduced grid corrosion, a lower black residue rating, longer discharge life, lower water loss, improved charge acceptance, longer life, etc.

### CROSS REFERENCE TO RELATED APPLICATIONS

This application is a 371 Application to PCT Application No. PCT/US2021/025796, filed Apr. 5, 2021, which claims priority to U.S. Provisional Pat. Application Serial No. 63/005,842, filed Apr. 6, 2020, which is hereby fully incorporated by reference herein.

### FIELD OF INVENTION

This application is directed to improved surfactant coated separators. Certain types, amounts, and blends of surfactants are used to create the improved surfactant coated separators.

### BACKGROUND

Automobile technology has soared over the last century, which has required batteries to evolve and perform under a very strict new set of guidelines. These new guidelines have led to many advances including Enhanced Flooded Batteries (EFB) and Idle-Stop-Start (ISS) batteries. The movement to both of these new types of batteries, as well as others, however have resulted in new issues relating to electrolyte volume loss, or “water loss” as it is commonly referred to in the industry. Water loss can occur for a variety of reasons in a lead-acid battery. One reason is overcharging, which causes electrolysis of water. The water loss affect can be further amplified by the use of carbon in the negative electrode. Carbon is a common component in the battery industry for controlling charge acceptance and sulfation.

To address these water loss issues, a certain class of nonionic surfactants have been used on the battery separator. See, for example, the disclosure of WO 2016/138369, which is assigned to Daramic LLC and is incorporated by reference herein in its entirety. It is believed that these nonionic surfactants act to lower water loss by increasing the overvoltage at which the electrolysis of water normally occurs. However, use of these nonionic surfactants do have some drawbacks. One drawback is that a drop in dynamic charge acceptance is observed when the nonionic surfactant is used. Without wishing to be bound by any particular theory, this is believed to be due to the surfactant forming a barrier on the negative electrode, which hinders charge acceptance. Another drawback is that an increase in dark colored residue formation is observed in batteries utilizing these nonionic surfactants. This residue is believed to be comprised of stearate and/or palmitate. Finally, it is believed that these nonionic surfactants may cause foaming when exposed to battery acid. Foaming is an issue for battery companies, particularly those using a two-step process to form their batteries. Acid foaming causes safety issues as the foam may seep out and also reduces visibility.

Thus, it would be desirable if the nonionic surfactants could be used to lower water loss and some of their drawbacks could be minimized or eliminated.

### SUMMARY

Described herein is a surfactant blend that effectively lowers water loss, while minimizing or eliminating the drawbacks associated with the use of nonionic surfactants. The surfactant blend herein contains nonionic surfactants and ionic surfactants, and when applied to a battery separator that is inserted into a battery, the resulting battery exhibits reduced water loss and improved oxidative resistance, while among other things the reduction in charge acceptance typically caused by nonionic surfactants is reduced or eliminated.

In one aspect, a battery separator is described and comprises the following: 1) a porous membrane; and 2) a surfactant coating that comprises a nonionic and an ionic surfactant. In some preferred embodiments, the basis weight of the nonionic surfactant on the porous membrane is 10 g/m² or less or 7 g/m² or less. In some preferred embodiments, the basis weight of the nonionic surfactant is from 1 g/m² to 5 g/m², from 1 g/m² to 4 g/m², or from 2.5 g/m² to 4 g/m². The ionic surfactant may be present on the porous membrane in an amount from 0.5 wt.% to 5.0 wt.% or from 1.0 wt. to 3.0 wt.% based on the weight of the separator.

In some preferred embodiments, the porous membrane may be a porous membrane comprising polyethylene. The porous membrane may be microporous. The porous membrane may be a porous membrane, a woven, a non-woven, or combinations of the foregoing.

The nonionic surfactant is not so limited and may be at least one selected from the following: fatty alcohols, cetyl alcohols, stearyl alcohols, pentaethylene glycol monododecyl ether, polyoxypropylene glycol alkyl ethers, polyoxyethylene glycol, octylphenol ethers, polyoxyethylene glycol alkyl ethers, octaethylene glycol monododecyl ether, polyoxyethylene glycol alkylphenol ethers, polyoxyethylene glycol sorbitan alkyle esters, oleyl alcohols, block copolymers of polyethylene glycol, block copolymers of polypropylene glycol, glucoside alkyl ethers, decyl glucoside, lauryl glucoside, octyl glucoside, nonoxynol-9, glycerol alkyl esters, polysorbates, sorbitan alkyl esters, glyceryl laurate, cocamide, costearyl alcohols, methallyl-capped non-ionic surfactant, polyol fatty acid esters, polyethyoxylated esters, polyethoxylated fatty alcohols, alkyl polysaccharides, alkyl polyglycosides, amine ethoxylates, sorbitan fatty acid ester ethoxylates, organosilicone based surfactants, ethylene vinyl acetate terpolymers, ethoxylated alkyl aryl phosphate esters, sucrose esters of fatty acids, polyethoxylated alcohols, polyethylene oxide, acid-soluble sugars, sucrose esters of fatty acids, organic fatty acids, hydroxyl acid, nonionic surfactant, octylphenol ethoxylate surfactant, octylphenol ethoxylate nonionic surfactant, and combinations thereof.

In some embodiments, the nonionic surfactant wherein the non-ionic surfactant has a cloud point rating greater than about 15° C., greater than about 20° C., or greater than about 25° C.

In some embodiments, the nonionic surfactant may have the following structures:

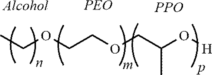

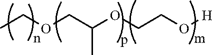

In the foregoing structures n may be an integer from 5 to 20 or from 9 to 17, m may be an integer from 1 to 15 or from 6 to 10, and p may be an integer from 0 to 10 or from 0 to 7.

The ionic surfactant may be a cationic surfactant, an anionic surfactant, or an amphoteric surfactant.

In some embodiments, the ionic surfactant may be at least one selected from the following: sulfates; alkyl sulfates; ammonium lauryl sulfates; sodium lauryl sulfates; alkyl ether sulfates; sodium laureth sulfate; sulfonates, docusates; dioctyl sodium sulfosuccinate; alkyl benzene sulfonates; phosphates; alkyl ether phosphates; carboxylates; alkyl carboxylates; fatty acid salts; sodium stearate; sodium lauroyl sarcosinate; Alkyltrimethylammonium; cetylpyridinium; polyethoxylated tallow amine; benzalkonium; benzethonium; dimethyldioctadecylammonium; dioctadecyldimethylammonium salts of alkyl sulfates; alkylarylsulfonate salts; alkylphenol-alkylene oxide addition products; soaps; alkyl-naphthalenesulfonate salts; one or more sulfo-succinates, such as an anionic sulfo-succinate; dialkyl esters of sulfo-succinate salts; amino compounds (primary, secondary or tertiary amines; quaternary amines; block copolymers of ethylene oxide and propylene oxide; various polyethylene oxides; salts of mono and dialkyl phosphate esters, and mixtures thereof.

In some embodiments, the ionic surfactant may be an anionic surfactant having the following structure:

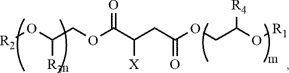

where n is an integer from 0 to 10, m is an integer from 0 to 10, R1 is H, a C1 to C10 linear or branched, saturated or unsaturated alkyl group, a C1 to C10 fatty alcohol, a C1 to C10 alcohol, or an aromatic group, and R2 is H, a C1 to C10 linear or branched, saturated or unsaturated alkyl group, a C1 to C10 linear or branched, saturated or unsaturated fatty alcohol, a C1 to C10 linear or branched, saturated or unsaturated alcohol, or an aromatic group, n and m are the same or different, R1 and R2 are the same or different, R3 is hydrogen or methyl, R4 is hydrogen or methyl, R3 and R4 are the same or different, X is a negatively charged groups such as SO3—, COO—, PO4-2, and the like. A positive counter ion to the anionic surfactant is also present and may be at least one of Na⁺, K⁺, Li⁺, NH₄⁺, Ca²⁺, Mg²⁺, and the like.

In some embodiments, the ionic surfactant may be an anionic surfactant having the following formula:

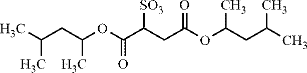

In another aspect, a battery separator described herein may exhibit at least one of the following: a perox80 value that is 90% or more, 91% or more, 92% or more, 93% or more, 94% or more, 95% or more, 96% or more, 97% or more, 98% or more, 99% or more or 100% of an initial value before the perox80 test was conducted; and an ERBOIL value that is less than 60.

In another aspect, a lead acid battery is described that comprises the following: 1) a battery separator as described herein above; and at least one grid comprising lead or a lead alloy, wherein the grid exhibits decreased grid corrosion compared to a battery where the battery separator is not as disclosed herein, i.e., does not have the surfactant blend described herein applied to a surface of the battery separator. For the ERBOIL test, a separator is subjected to 10 minutes in boiling water followed by a 20 minute soak in sulfuric acid. After this, the ER is measured.

In another aspect, a lead acid battery is described where the lead acid battery comprises a battery separator as described herein and the battery has a black residue rating less than 3 or less than 2.

In another aspect, a lead acid battery is described where the lead acid battery comprises a battery separator as described herein and the battery exhibits improved partial state of charge (PSOC) cycle life testing. PSOC cycle life testing is compared to a battery where the separator does not comprise a surfactant blend as described herein.

In another aspect, a battery separator is described herein that comprises the following: a porous membrane and a surfactant coating that comprises a nonionic surfactant on at least one side of the porous membrane, wherein the coating weight of the non-ionic surfactant is from 1 g/m² to 5 g/m². The coating weight of the non-ionic surfactant may also be from 2 g/m² to 4 g/m², from 3 g/m² to 4 g/m². This battery separator may exhibit at least one of the following: longer discharge life, less water loss, improved charge acceptance, and longer life compared to a battery comprising a separator with a higher coating weight of non-ionic surfactant.

In another aspect, a battery separator comprising a porous membrane and a surfactant coating is described. In this embodiment, the surfactant of the surfactant coating consists of a compound having the following structures:

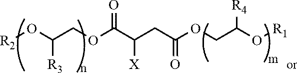

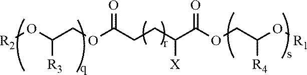

where n is an integer from 0 to 10, m is an integer from 0 to 10, n and m are the same or different, R1 is H, a C1 to C10 linear or branched, saturated or unsaturated alkyl group, a C1 to C10 fatty alcohol, a C1 to C10 alcohol, or an aromatic group, and R2 is H, a C1 to C10 linear or branched, saturated or unsaturated alkyl group, a C1 to C10 linear or branched, saturated or unsaturated fatty alcohol, a C1 to C10 linear or branched, saturated or unsaturated alcohol, or an aromatic group, n and m are the same or different, R1 and R2 are the same or different, R3 is hydrogen or methyl or a C1 to C5 alkyl group, R4 is hydrogen or methyl or a C1 to C5 alkyl group R3 and R4 are the same or different, and X is a negatively charged groups such as SO3—, COO—, PO4-2, and the like; and also a positively charged counter-ion. In some embodiments, R3 and R4 are the same and are both hydrogen. In some embodiments, R3 and R4 are the same and both methyl groups. In some embodiments, X is SO3—. In some embodiments, X is COO—. In some embodiments, X is PO4-2. In some embodiments, m and n are each an integer from 1 to 5 or are each integers from 6 to 10. In some embodiments, n is an integer from 1 to 5 or an integer from 6 to 10. In some embodiments, m is an integer from 1 to 5 or an integer from 6 to 10. In some embodiments, q is an integer from 1to 10, 1 to 5 or 6 to 10. In some embodiments, r is an integer from 1 to 10, 1 to 5, or 6 to 10. In some embodiments, s is an integer from 1 to 10, 1 to 5, or 6 to 10. In some embodiments, the surfactant has the following structure:

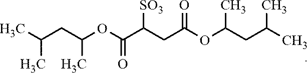

### DESCRIPTION OF DRAWINGS

FIG. 1 is a graph comparing black residue ratings of embodiments described herein.

FIG. 2 is an image showing grid corrosion for some embodiments described herein.

FIG. 3 is a graph showing loss of grid mass after a specified period of time for embodiments disclosed herein.

### DETAILED DESCRIPTION

Described herein is a surfactant blend that effectively lowers water loss, while minimizing or eliminating the drawbacks associated with the use of nonionic surfactants. The surfactant blend herein contains nonionic surfactants and ionic surfactants, and when applied to a battery separator that is inserted into a battery, the resulting battery exhibits reduced water loss and improved oxidative resistance, while among other things the reduction in charge acceptance typically caused by nonionic surfactants is reduced or eliminated.

### Battery Separator 1

In one aspect, a battery separator is described and comprises the following: 1) a porous membrane; and 2) a surfactant coating that comprises a nonionic and an ionic surfactant. In some preferred embodiments, the basis weight of the nonionic surfactant on the porous membrane is 10 g/m² or less or 7 g/m² or less. In some preferred embodiments, the basis weight of the nonionic surfactant is from 1 g/m² to 5 g/m², from 1 g/m² to 4 g/m², or from 2.5 g/m² to 4 g/m². The ionic surfactant may be present on the porous membrane in an amount from 0.5 wt.% to 5.0 wt.% or from 1.0 wt. to 3.0 wt.% based on the weight of the separator.

### Porous Membrane

In some preferred embodiments, the porous membrane may be microporous, macroporous, mesoporous, or nanoporous. In some preferred embodiments, the average pore size of the porous membrane is one micron or less.

The composition of the porous membrane is not so limited, and may include polymers or not include polymers.

If the porous membrane is polymeric, it may have a composition that includes at least one of the polymers, thermoplastic polymers, polyvinyl chlorides (“PVCs”), phenolic resins, natural or synthetic rubbers, synthetic wood pulp, lignins, glass fibers, synthetic fibers, cellulosic fibers, and/or combinations thereof. The natural or synthetic rubbers may include one or more of rubber, latex, natural rubber, synthetic rubber, cross-linked or uncross-linked natural or synthetic rubbers, cured or uncured rubbers, crumb or ground rubber, polyisoprenes, methyl rubber, polybutadiene, chloroprene rubbers, butyl rubber, bromobutyl rubber, polyurethane rubber, epichlorhydrin rubber, polysulphide rubber, chlorosulphonyl polyethylene, polynorbornene rubber, acrylate rubber, fluorine rubber and silicone rubber and copolymer rubbers, such as styrene/butadiene rubbers, acrylonitrile/butadiene rubbers, ethylene/propylene rubbers (“EPM” and “EPDM”) and ethylene/vinyl acetate rubbers, and/or combinations thereof.

In some aspects of the present invention, the porous membrane’s composition may further possess a filler. In some embodiments, that filler is at least one of silica, dry finely divided silica, precipitated silica, amorphous silica, highly friable silica, alumina, talc, fish meal, fish bone meal, barium sulfate (BaSO₄), carbon, conductive carbon, graphite, artificial graphite, activated carbon, carbon paper, acetylene black, carbon black, high surface area carbon black, graphene, high surface area graphene, keitjen black, carbon fibers, carbon filaments, carbon nanotubes, open-cell carbon foam, a carbon mat, carbon felt, carbon Buckminsterfullerene (“Bucky Balls”), an aqueous carbon suspension, flake graphite, oxidized carbon, and/or combinations thereof.

In some embodiments, the composition of the porous membrane may further comprise a processing oil left over from manufacture of the substrate. One benefit of the battery separator described herein is the ability to reduce processing oil content in the substrate below 20%, below 15%, below 10%, or below 5%. For example, the processing oil content may be reduced as low as 1% or less, 2% or less, 3% or less, 4% or less, 5% or less, 6% or less, 7% or less, 8% or less, 9% or less, 10% or less, 11% or less, 12% or less, 13% or less, 14% or less, 15% or less, 16% or less, 17% or less, 18% or less, 19% or less, or 20% or less. Conventionally, significant amounts of processing oil was left behind was to, among other things, improve oxidation resistance. However, with the addition of the material layer on at least one surface of the polymeric substrate in the battery separator described herein, the concern of oxidation resistance of the substrate is lower and amounts of remaining processing oil in the substrate can be reduced. Reducing the amount of processing oil can have the positive effect of increasing ionic conductivity of the substrate and/or lowering the electrical resistance across the substrate. Thus, the ability to have lower amounts of remaining processing oil in the substrate is significant and may lead to improved separator performance. Although the ability to lower processing oil content of the substrate is a benefit made possible by the structure of the improved battery separator described herein, embodiments of the battery separator where the substrate has a processing oil content above 20% are also workable and have other benefits.

In some preferred embodiments, the porous membrane may be a porous membrane comprising polyethylene. The porous membrane may be microporous. The porous membrane may be a porous polyolefin membrane, a filled polyolefin porous membrane, an absorptive glass mat (AGM), a woven, a non-woven, or combinations of the foregoing. One possible combination is the combination of an AGM with a filled, e.g., silica-filled, polyolefin porous membrane.

In some preferred embodiments, the porous membrane is a filled polyolefin porous membrane, e.g., like the silica-filled polyethylene products typically sold by Daramic LLC. In some other preferred embodiments, the porous membrane may be an absorptive glass mat (AGM

In some embodiments, one or more surface or face of the porous membrane may have ribs, protrusions, or both ribs and protrusions. In embodiments where ribs are present, the ribs do not have any particular structure but the may be at least one of the following: continuous ribs, discontinuous ribs, longitudinally extending ribs, latitudinally extending ribs, diagonally extending ribs, integral ribs, non-integral ribs, mini ribs, and combinations thereof. For example, the ribs could be discontinuous and diagonally extending ribs. Protrusions are not ribs. One example of a protrusions may include, but is not limited to, dimples. When ribs, protrusions, or ribs and protrusions are formed on both faces of the substrate, the types of ribs, protrusions, or ribs and protrusions formed on each face or surface may be the same or different. For example, lattitudinally extending ribs may be formed on one face or surface of the substrate and longitudinally extending ribs may be formed on the other face or surface. In some preferred embodiments, lattitudinally extending ribs may be formed on a positive face of the porous membrane, and longitudinally extending ribs may be formed on a negative face (i.e., negative cross ribs).

In some embodiments when ribs, protrusions, or ribs and protrusions are formed on a surface of the substrate, one or more edge regions of the substrate may not include ribs, protrusions, or ribs and protrusions or the one or more edge regions may only include mini ribs, mini protrusions, or mini ribs and protrusions. A mini rib or mini protrusion may have a maximum height from the face of the substrate to the highest point of the rib or protrusion that is at most 100 to at most 250 microns from the face of the substrate. In some embodiments, the maximum height may be at most 75 microns, at most 50 microns, at most 25 microns, at most 125 microns, at most 150 microns, at most 175 microns, at most 200 microns, or at most 225 microns. This type of structure may be useful if the final structure of the battery separator is a pouch or sleeve that involves welding of the edges of the substrate material to form. In such embodiments where regions with no ribs or protrusions (or only mini ribs or protrusions) are formed, it is preferred that no material layer be formed in these regions either.

In some embodiments, the thickness of the substrate may be in the range of 50 to 500 microns, 75 to 500 microns, 100 to 500 microns, 125 to 500 microns, 150 to 500 microns, 175 to 500 microns, 200 to 500 microns, 225 to 500 microns, 250 to 500 microns, 300 to 500 microns, 325 to 500 microns, 350 to 500 microns, 375 to 500 microns, 400 to 500 microns, 425 to 500 microns, 450 to 500 microns, or 475 to 500 microns. In embodiments, where ribs are formed on one or more surfaces of the substrate, the thickness of the substrate is the thickness of what is often referred to the backweb, which is the substrate not considering the height of the ribs formed thereon.

### Surfactant Coating

The surfactant coating comprises a nonionic and an ionic surfactant. In some preferred embodiments, the basis weight of the nonionic surfactant on the porous membrane is 10 g/m² or less, 9 g/m² or less, 8 g/m² or less, 7 g/m² or less, 6 g/m² or less, 5 g/m² or less, 4 g/m² or less, 3 g/m² or less, 2 g/m² or less, or 1 g/m² or less. In some preferred embodiments, the basis weight of the nonionic surfactant is from 1 g/m² to 5 g/m², from 1 g/m² to 4 g/m², from 1 g/m² to 3 g/m² , from 2 g/m² to 4 g/m², from 2.5 g/m² to 4 g/m², or from 3 g/m² to 4 g/m². The ionic surfactant may be present on the porous membrane in an amount from 0.5 wt.% to 5.0 wt.%, from 0.5 wt.% to 4.0 wt.%, from 0.5 wt.% to 3.0 wt.%, from 1.0 wt.% to 4.0 wt.%, from 1.0 wt. to 3.0 wt.%, or from 1.0 wt.% to 2.0 wt.% based on the weight of the separator.

The nonionic surfactant is not so limited. It may be at least one selected from the following: fatty alcohols, cetyl alcohols, stearyl alcohols, pentaethylene glycol monododecyl ether, polyoxypropylene glycol alkyl ethers, polyoxyethylene glycol, octylphenol ethers, polyoxyethylene glycol alkyl ethers, octaethylene glycol monododecyl ether, polyoxyethylene glycol alkylphenol ethers, polyoxyethylene glycol sorbitan alkyle esters, oleyl alcohols, block copolymers of polyethylene glycol, block copolymers of polypropylene glycol, glucoside alkyl ethers, decyl glucoside, lauryl glucoside, octyl glucoside, nonoxynol-9, glycerol alkyl esters, polysorbates, sorbitan alkyl esters, glyceryl laurate, cocamide, costearyl alcohols, methallyl-capped non-ionic surfactant, polyol fatty acid esters, polyethyoxylated esters, polyethoxylated fatty alcohols, alkyl polysaccharides, alkyl polyglycosides, amine ethoxylates, sorbitan fatty acid ester ethoxylates, organosilicone based surfactants, ethylene vinyl acetate terpolymers, ethoxylated alkyl aryl phosphate esters, sucrose esters of fatty acids, polyethoxylated alcohols, polyethylene oxide, acid-soluble sugars, sucrose esters of fatty acids, organic fatty acids, hydroxyl acid, nonionic surfactant, octylphenol ethoxylate surfactant, octylphenol ethoxylate nonionic surfactant, and combinations thereof.

In some embodiments, the nonionic surfactant wherein the non-ionic surfactant has a cloud point rating greater than about 15° C., greater than about 20° C., or greater than about 25° C.

In some embodiments, the nonionic surfactant may have the following structures:

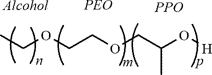

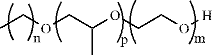

In the foregoing structures n may be an integer from 5 to 20, 6 to 20, 7 to 20, 8 to 20, 9 to 20, 10 to 20, 11 to 20, 12 to 20, 13 to 20, 14 to 20, 15 to 20, 16 to 20, 17 to 20, 18 to 20, 19 to 20, or from 9 to 17, m may be an integer from 1 to 15, 2 to 15, 3 to 15, 4 to 15, 5 to 15, 6 to 15, 7 to 15, 8 to 15, 9 to 15, 10 to 15, 11 to 15, 12 to 15, 13 to 15, 14 to 15, or from 6 to 10, and p may be an integer from 0 to 10, 0 to 9, 0 to 8, 0 to 7, 0 to 6 0 to 5, 0 to 4, 0 to 3, 0 to 2, or 0 to 1, or from 0 to 7.

The ionic surfactant may be a cationic surfactant, an anionic surfactant, or an amphoteric surfactant.

In some embodiments, the ionic surfactant may be at least one selected from the following: sulfates; alkyl sulfates; ammonium lauryl sulfates; sodium lauryl sulfates; alkyl ether sulfates; sodium laureth sulfate; sulfonates, docusates; dioctyl sodium sulfosuccinate; alkyl benzene sulfonates; phosphates; alkyl ether phosphates; carboxylates; alkyl carboxylates; fatty acid salts; sodium stearate; sodium lauroyl sarcosinate; Alkyltrimethylammonium; cetylpyridinium; polyethoxylated tallow amine; benzalkonium; benzethonium; dimethyldioctadecylammonium; dioctadecyldimethylammonium salts of alkyl sulfates; alkylarylsulfonate salts; alkylphenol-alkylene oxide addition products; soaps; alkyl-naphthalenesulfonate salts; one or more sulfo-succinates, such as an anionic sulfo-succinate; dialkyl esters of sulfo-succinate salts; amino compounds (primary, secondary or tertiary amines; quaternary amines; block copolymers of ethylene oxide and propylene oxide; various polyethylene oxides; salts of mono and dialkyl phosphate esters, and mixtures thereof.

In some embodiments, the ionic surfactant may be an anionic surfactant having the following structure:

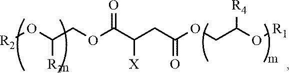

where n is an integer from 0 to 10, 0 to 9, 0 to 8, 0 to 7, 0 to 6, 0 to 5, 0 to 4, 0 to 3, 0 to 2, or 0 to 1, m is an integer from 0 to 10, 0 to 9, 0 to 8, 0 to 7, 0 to 6, 0 to 5, 0 to 4, 0 to 3, 0 to 2, or 0 to 1 R1 is H, a C1 to C10, C1 to C9, C1 to C8, C1 to C7, C1 to C6, C1 to C5, C1 to C4, C1 to C3, or C1 to C2 linear or branched, saturated or unsaturated alkyl group, a C1 to C10, C1 to C9, C1 to C8, C1 to C7, C1 to C6, C1 to C5, C1 to C4, C1 to C3, or C1 to C2 fatty alcohol, a C1 to C10, C1 to C9, C1 to C8, C1 to C7, C1 to C6, C1 to C5, C1 to C4, C1 to C3, or C1 to C2 alcohol, or an aromatic group, and R2 is H, a C1 to C10, C1 to C9, C1 to C8, C1 to C7, C1 to C6, C1 to C5, C1 to C4, C1 to C3, or C1 to C2 linear or branched, saturated or unsaturated alkyl group, a C1 to C10, C1 to C9, C1 to C8, C1 to C7, C1 to C6, C1 to C5, C1 to C4, C1 to C3, or C1 to C2 linear or branched, saturated or unsaturated fatty alcohol, a C1 to C10, C1 to C9, C1 to C8, C1 to C7, C1 to C6, C1 to C5, C1 to C4, C1 to C3, or C1 to C2 linear or branched, saturated or unsaturated alcohol, or an aromatic group, n and m are the same or different, R1 and R2 are the same or different, R3 is hydrogen or methyl, R4 is hydrogen or methyl, R3 and R4 are the same or different, X is a negatively charged groups such as SO3—, COO—, PO4-2, and the like. A positive counter ion to the anionic surfactant is also present and may be at least one of Na⁺, K⁺, Li⁺, NH₄⁺, Ca²⁺, Mg²⁺, and the like.

In some embodiments, the ionic surfactant may be an anionic surfactant having the following formula:

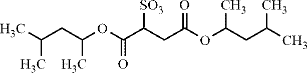

In another aspect, a battery separator described herein may exhibit at least one of the following: a perox80 value that is 90% or more, 91% or more, 92% or more, 93% or more, 94% or more, 95% or more, 96% or more, 97% or more, 98% or more, 99% or more or 100% of an initial value before the perox80 test was conducted; and an ERBOIL value that is less than 60, less than 50, less than 40, less than 30, less than 20, less than 10, or less than 5, less than 1.

### Lead Acid Battery

In another aspect, a lead acid battery is described that comprises the following: 1) a battery separator as described herein above; and at least one grid comprising lead or a lead alloy, wherein the grid exhibits decreased grid corrosion compared to a battery where the battery separator is not as disclosed herein, i.e., does not have the surfactant blend described herein applied to a surface of the battery separator. For the ERBOIL test, a separator is subjected to 10 minutes in boiling water followed by a 20 minute soak in sulfuric acid. After this, the ER is measured.

In another aspect, a lead acid battery is described where the lead acid battery comprises a battery separator as described herein and the battery has a black residue rating less than 3, less than 2, or less than 1.

In another aspect, a lead acid battery is described where the lead acid battery comprises a battery separator as described herein and the battery exhibits improved partial state of charge (PSOC) cycle life testing. PSOC cycle life testing is compared to a battery where the separator does not comprise a surfactant blend as described herein.

### Battery Separator 2

In another aspect, a battery separator is described herein that comprises the following: a porous membrane and a surfactant coating that comprises a nonionic surfactant on at least one side of the porous membrane, wherein the coating weight of the non-ionic surfactant is from 1 g/m² to 5 g/m². The coating weight of the non-ionic surfactant may also be from 2 g/m² to 4 g/m², from 3 g/m² to 4 g/m². The surfactant coating may also comprise an ionic surfactant as described hereinabove. A battery comprising this battery separator may exhibit at least one of the following: longer discharge life, less water loss, improved charge acceptance, and longer life compared to a battery comprising a separator with a higher coating weight of non-ionic surfactant.

### Battery Separator 3

In another aspect, a battery separator comprising a porous membrane and a surfactant coating is described. In this embodiment, the surfactant of the surfactant coating comprises, consists of, or consists essentially of a compound having the following structures:

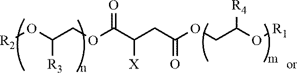

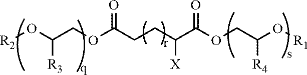

where n is an integer from 0 to 10, 0 to 9, 0 to 8, 0 to 7, 0 to 6, 0 to 5, 0 to 4, 0 to 3, 0 to 2, or 0 to 1, m is an integer from 0 to 10, 0 to 9, 0 to 8, 0 to 7, 0 to 6, 0 to 5, 0 to 4, 0 to 3, 0 to 2, or 0 to 1, n and m are the same or different, R1 is H, a C1 to C10, C1 to C9, C1 to C8, C1 to C7, C1 to C6, C1 to C5, C1 to C4, C1 to C3, or C1 to C2 linear or branched, saturated or unsaturated alkyl group, a C1 to C10, C1 to C9, C1 to C8, C1 to C7, C1 to C6, C1 to C5, C1 to C4, C1 to C3, or C1 to C2 fatty alcohol, a C1 to C10, C1 to C9, C1 to C8, C1 to C7, C1 to C6, C1 to C5, C1 to C4, C1 to C3, or C1 to C2 alcohol, or an aromatic group, and R2 is H, a C1 to C10, C1 to C9, C1 to C8, C1 to C7, C1 to C6, C1 to C5, C1 to C4, C1 to C3, or C1 to C2 linear or branched, saturated or unsaturated alkyl group, a C1 to C10, C1 to C9, C1 to C8, C1 to C7, C1 to C6, C1 to C5, C1 to C4, C1 to C3, or C1 to C2 linear or branched, saturated or unsaturated fatty alcohol, a C1 to C10, C1 to C9, C1 to C8, C1 to C7, C1 to C6, C1 to C5, C1 to C4, C1 to C3, or C1 to C2 linear or branched, saturated or unsaturated alcohol, or an aromatic group, n and m are the same or different, R1 and R2 are the same or different, R3 is hydrogen or methyl or a C1 to C5, C1 to C4, C1 to C3, or C1 to C2 alkyl group, R4 is hydrogen or methyl or a C1 to C5, C1 to C4, C1 to C3, or C1 to C2 alkyl group R3 and R4 are the same or different, and X is a negatively charged groups such as SO3—, COO—, PO4-2, and the like; and also a positively charged counter-ion. In some embodiments, R3 and R4 are the same and are both hydrogen. In some embodiments, R3 and R4 are the same and both methyl groups. In some embodiments, X is SO3—. In some embodiments, X is COO—. In some embodiments, X is PO4-2. In some embodiments, m and n are each an integer from 1 to 5, 1 to 4, 1 to 3, or 1 to 2 or are each integers from 6 to 10, 6 to 9, 6 to 8, or 6 to 7. In some embodiments, n is an integer from 1 to 5, 1 to 4, 1 to 3, or 1 to 2 or an integer from 6 to 10, 6 to 9, 6 to 8, or 6 to 7. In some embodiments, m is an integer from 1 to 5, 1 to 4, 1 to 3, or 1 to 2 or an integer from 6 to 10, 6 to 9, 6 to 8, or 6 to 7. In some embodiments, q is an integer from 1 to 10, 1 to 9, 1 to 8, 1 to 7, 1 to 6, 1 to 5, 1 to 4, 1 to 3, 1 to 2 or 6 to 10, 7 to 10, 8 to 10, or 9 to 10. In some embodiments, r is an integer from 1 to 10, 1 to 9, 1 to 8, 1 to 7, 1 to 6, 1 to 5, 1 to 4, 1 to 3, 1 to 2, or 6 to 10, 6 to 9, 6 to 8, or 6 to 7. In some embodiments, s is an integer from 1 to 10, 1 to 9, 1 to 8, 1 to 7, 1 to 6, 1 to 5, 1 to 4, 1 to 3, or 1 to 2 or 6 to 10, 6 to 9, 6 to 8, or 6 to 7. In some embodiments, the surfactant has the following structure:

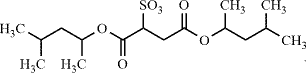

### EXAMPLES

Examples were prepared using a nonionic surfactant having PPO and PEO blocks such as the following:

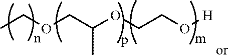

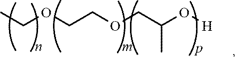

wherein n may be an integer from 12 to 15, m may be an integer from 1 to 15 or from 6 to 10, and p may be an integer from 0 to 10 or from 0 to 7 wherein n is, m is, and p is. Low, medium, and high amounts of surfactant were applied to the same type of Daramic® separator. These Examples were evaluated. A control using that same type of Daramic® separator, but no non-ionic surfactant, was also evaluated. Results of the evaluation are found in Table 1 below:

TABLE 1

| Non-ionic Surfactant Level | Cold Crank @-17° C., 10 sec(V) | Water Loss @70C (g/Ah) | Charge Acceptance (A/Ah) | Partial State of Charge Cycle (Cycle) |
|---|---|---|---|---|
| Control A (0 g/m²) | 7.9 | 6.0 | 0.80 | 50000 |
| Example 1-Low (3.5 g/m²) | 7.9 | 4.3 | 0.78 | 49000 |
| Example 2-Medium (5.5 g/m²) | 7.7 | 4.4 | 0.71 | 9000 |
| Example 3-High (7.5 g/m²) | 7.5 | 4.8 | 0.65 | 5000 |

Cold Crank and Water Loss test are based on EN 50342-1:2015. Charge acceptance and Partial State of Charge Cycle are based on SBA S0101 (2014).

Thus, the inventors have found that use or addition of low levels of non-ionic surfactant result in unexpectedly improved battery properties compared to the battery properties for control and medium and high non-ionic surfactant amounts. Low levels of non-ionic surfactant are believed to be less than 5 g/m² and more than 1 g/m², but may be from 2 g/m² to 4 g/m², or from 3 g/m² to 4 g/m². These improved properties include higher partial state of charge cycles, improved charge acceptance, and lower water loss.

Examples were prepared using a blend of non-ionic surfactant with an anionic surfactant having the following structure:

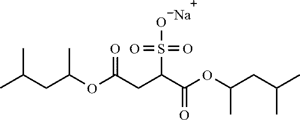

A control was also prepared using non-ionic surfactant only. In each example, the surfactant or surfactant blend was coated onto the same Daramic® separator. The Examples are shown in Table 2.

TABLE 2

| Separator attribute | Non-ionic Surfactant only (Control B) | Non-ionic surfactant + anionic surfactant (Example 4) | Non-ionic surfactant + anionic surfactant (Example 5) |
|---|---|---|---|
| Weight of anionic surfactant (wt%) | 0.1±0.0 | at least about 1% anionic surfactant | At least about 1% anionic surfactant |
| Basis weight of nonionic surfactant | 7.3± 0.4 | 6.6±0.1 | 6.9±0.1 |
| Overall thickness (micron) | 399±5 | 401±1 | 401±2 |
| Total oil content (%) | 16.7±0.5 | 16.7±0.1 | 16.4±0.1 |
| Porosity (%) | 63.5±0.1 | 63.0±0.8 | 63.1±0.3 |
| ER (10/20 min) (mΩ·cm²) | 76±1 | 78±3 | 80±1 |
| Puncture Resistance (N) | 11.9±0.5 | 14.7±1.3 | 15.5±0.9 |
| CMD elongation (%) | 461±42 | 486±48 | 483±45 |
| Oxidation resistance 20h (%) | 451±38 | 468±90 | 455±31 |
| Oxidation resistance 40h (%) | 405±35 | 450±51 | 437±46 |
| A-dimensional stability MD at 77C (%) | 0.4 | 0.6 | 0.5 |
| ER BOIL (BOIL ER) | 63 | Not measured | 56 |
| PEROX80 | 384 | Not measured | 404 |

For an ER (10/20), the separator is boiled for 10 minutes in water and then soaked in sulfuric acid 1.28% +/- 0.005.

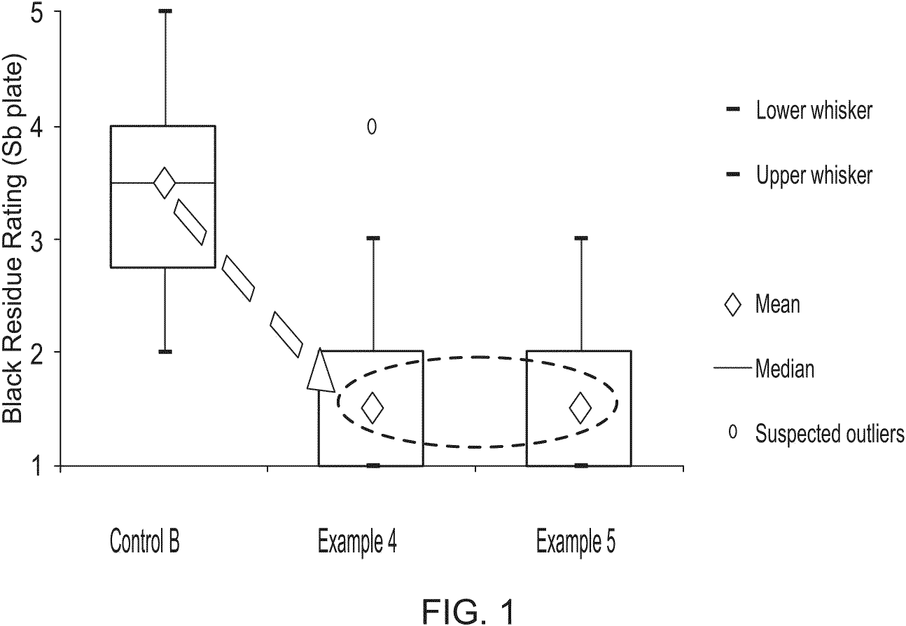

Addition of anionic surfactant to the nonionic surfactant was found to reduce black residue rating as shown in FIG. 1. This is significant because a reduction in black residue can improve battery safety by improving the ability of one to properly maintain the battery. When there is excessive buildup of black residue in the battery, this residue can block the “magic eye,” which is a part of a battery that informs the operater when maintenance is needed. It was found that the addition of at least about 1% anionic surfactant sufficient to get the good results mentioned above.

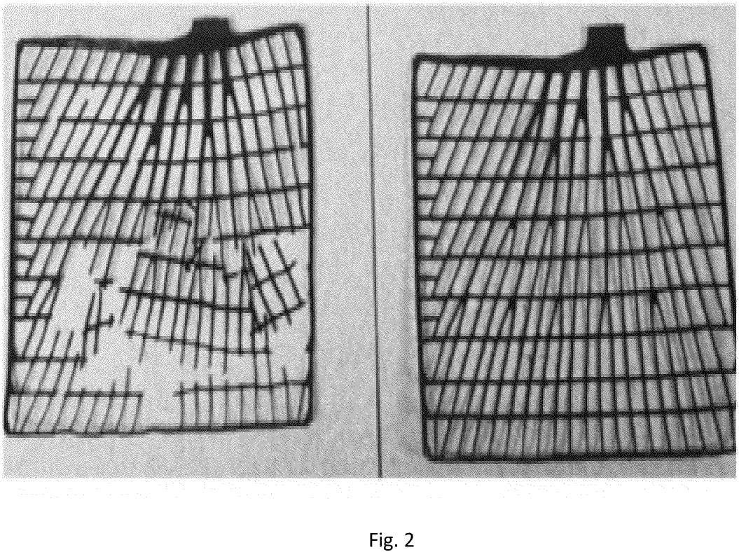

Addition of an anionic surfactant also helps with grid corrosion. This is shown in the image in FIG. 2 where the grid on the left was present in a battery comprising a separator like control B and the grid on the right was present in a battery comprising a separator like Example 4 or Example 5

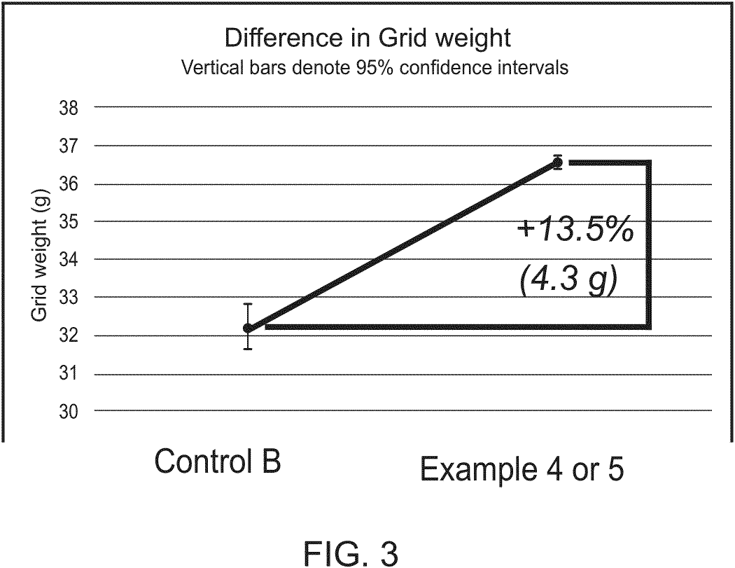

As shown in the graph in FIG. 3, plates used in a battery with a separator like that in Example 4 or Example 5, lost 13.5 % less grid mass at the end of life @ 12 weeks J280.

Example 6 and 7 are formed to include a surfactant coating on a Daramic® separator. In each surfactant coating, the surfactant consists of a single surfactant having one of the following structures or consists of two or more surfactants each having one of the following structures:

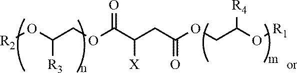

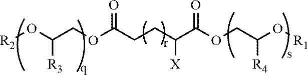

where n is an integer from 0 to 10, m is an integer from 0 to 10, n and m are the same or different, R1 is H, a C1 to C10 linear or branched, saturated or unsaturated alkyl group, a C1 to C10 fatty alcohol, a C1 to C10 alcohol, or an aromatic group, and R2 is H, a C1 to C10 linear or branched, saturated or unsaturated alkyl group, a C1 to C10 linear or branched, saturated or unsaturated fatty alcohol, a C1 to C10 linear or branched, saturated or unsaturated alcohol, or an aromatic group, n and m are the same or different, R1 and R2 are the same or different, R3 is hydrogen or methyl or a C1 to C5 alkyl group, R4 is hydrogen or methyl or a C1 to C5 alkyl group R3 and R4 are the same or different, and X is a negatively charged groups such as SO3—, COO—, PO4-2, and the like; and also a positively charged counter-ion. In some embodiments, R3 and R4 are the same and are both hydrogen. In some embodiments, R3 and R4 are the same and both methyl groups. In some embodiments, X is SO3—. In some embodiments, X is COO—. In some embodiments, X is PO4-2. In some embodiments, m and n are each an integer from 1 to 5 or are each integers from 6 to 10. In some embodiments, n is an integer from 1 to 5 or an integer from 6 to 10. In some embodiments, m is an integer from 1 to 5 or an integer from 6 to 10. In some embodiments, q is an integer from 1to 10, 1 to 5 or 6 to 10. In some embodiments, r is an integer from 1 to 10, 1 to 5, or 6 to 10. In some embodiments, s is an integer from 1 to 10, 1 to 5, or 6 to 10.

1. A battery separator comprising:
a porous membrane; and
a surfactant, which comprises a mixture of at least one ionic surfactant and at least one non-ionic surfactant, on at least one side of the porous membrane.

2. The battery separator of claim 1, wherein the at least one non-ionic surfactant is at least one selected from fatty alcohols, cetyl alcohols, stearyl alcohols, pentaethylene glycol monododecyl ether, polyoxypropylene glycol alkyl ethers, polyoxyethylene glycol, octylphenol ethers, polyoxyethylene glycol alkyl ethers, octaethylene glycol monododecyl ether, polyethylene glycol alkylphenol ethers, polyoxyethylene glycol sorbitan alkyle esters, oleyl alcohols, block copolymers of polyethylene glycol, block copolymers of polypropylene glycol, glucoside alkyl ethers, decyl glucoside, lauryl glucoside, octyl glucoside, nonoxynol-9, glycerol alkyl esters, polysorbates, sorbitan alkyl esters, glyceryl laurate, cocamide, costearyl alcohols, methallyl-capped non-ionic surfactant, polyol fatty acid esters, polyethyoxylated esters, polyethoxylated fatty alcohols, alkyl polysaccharides, alkyl polyglycosides, amine ethoxylates, sorbitan fatty acid ester ethoxylates, organosilicone based surfactants, ethylene vinyl acetate terpolymers, ethoxylated alkyl aryl phosphate esters, sucrose esters of fatty acids, polyethoxylated alcohols, polyethylene oxide, acid-soluble sugars, sucrose esters of fatty acids, organic fatty acids, hydroxyl acid, nonionic surfactant, octylphenol ethoxylate surfactant, octylphenol ethoxylate nonionic surfactant, and combinations thereof.

3. The battery separator of claim 1, wherein the non-ionic surfactant has a cloud point rating greater than about 15° C., greater than about 20° C., or greater than about 25° C.

4. The battery separator of claim 1, wherein the at least one non-ionic surfactant comprises a molecule with the following structure: 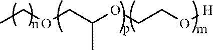
or 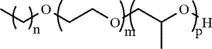
wherein n may be an integer from 5 to 20 or from 9 to 17, m may be an integer from 1 to 15 or from 6 to 10, and p may be an integer from 0 to 10 or from 0 to 7.

5. The battery separator of claim 1, wherein the at least one ionic surfactant is an anionic surfactant.

6. The battery separator of claim 1, wherein the ionic surfactant is a cationic surfactant, for example, selected from a group of alkoxylated ammonium salts.

7. The battery separator of claim 1, wherein the ionic surfactant is an amphoteric surfactant, for example, selected from a group of alkoxylated amino acids.

8. The battery separator of claim 1, wherein the ionic surfactant is at least one selected from: sulfates; alkyl sulfates; ammonium lauryl sulfates; sodium lauryl sulfates; alkyl ether sulfates; sodium laureth sulfate; sulfonates, docusates; dioctyl sodium sulfosuccinate; alkyl benzene sulfonates; phosphates; alkyl ether phosphates; carboxylates; alkyl carboxylates; fatty acid salts; sodium stearate; sodium lauroyl sarcosinate; Alkyltrimethylammonium; cetylpyridinium; polyethoxylated tallow amine; benzalkonium; benzethonium; dimethyldioctadecylammonium; dioctadecyldimethylammonium salts of alkyl sulfates; alkylarylsulfonate salts; alkylphenol-alkylene oxide addition products; soaps; alkyl-naphthalenesulfonate salts; one or more sulfo-succinates, such as an anionic sulfo-succinate; dialkyl esters of sulfo-succinate salts; amino compounds (primary, secondary or tertiary amines; quaternary amines; block copolymers of ethylene oxide and propylene oxide; various polyethylene oxides; salts of mono and dialkyl phosphate esters, and mixtures thereof.

9. The battery separator of claim 1, wherein the ionic surfactant has one of the following structures: 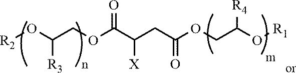 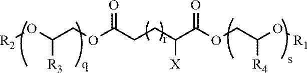
where n is an integer from 0 to 10, m is an integer from 0 to 10, n and m are the same or different, q is an integer from 0 to 10, r is an integer from 0 to 10, s is an integer from 0 to 10, and q, r, and s are the same or different, R1 is H, a C1 to C10 linear or branched, saturated or unsaturated alkyl group, a C1 to C10 fatty alcohol, a C1 to C10 alcohol, or an aromatic group, and R2 is H, a C1 to C10 linear or branched, saturated or unsaturated alkyl group, a C1 to C10 linear or branched, saturated or unsaturated fatty alcohol, a C1 to C10 linear or branched, saturated or unsaturated alcohol, or an aromatic group, n and m are the same or different, R1 and R2 are the same or different, R3 is hydrogen or methyl or a C1 to C5 alkyl group, R4 is hydrogen or methyl or a C1 to C5 alkyl group R3 and R4 are the same or different, and X is a negatively charged groups such as SO3—, COO—, PO4-2, and the like; and also a positively charged counter-ion.

10. The battery separator of claim 1, wherein the ionic surfactant has the following structure: 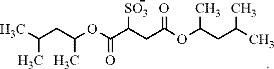
with a positively charged counter-ion that is optionally Na+.

11. The battery separator of claim 1, wherein the ionic surfactant is added in an amount of from 0.5 to 5.0 wt. %.

12. The battery separator of claim 11, wherein the ionic surfactant is added in an amount from 1.0 to 3.0 wt.%.

13. The battery separator of claim 1, wherein a non-ionic surfactant level is less than 10 g/m².

14. The battery separator of claim 13, wherein a the non-ionic surfactant level is less than 7 g/m².

15. The battery separator of claim 13, wherein the non-ionic surfactant level is from 1 g/m² to 5 g/m², from 1 g/m² to 4 g/m², or from 2.5 g/m² to 4 g/m².

16. The battery separator of claim 1, wherein the porous membrane is a porous membrane comprising polyethylene.

17. The battery separator of claim 1, wherein the battery separator exhibits at least one of the following:
a perox80 value that is 90% or more, 91% or more, 92% or more, 93% or more, 94% or more, 95% or more, 96% or more, 97% or more, 98% or more, 99% or more or 100% of an initial value before the perox80 test was conducted; and
an ERBOIL, value less than 60.

18. A lead acid battery comprising:
the battery separator according to claim 1; and
at least one grid comprising lead or a lead alloy, wherein the grid exhibits reduced grid corrosion compared to the same battery where the separator includes a coating with a non-ionic surfactant, but no ionic surfactant.

19. A lead acid battery comprising the battery separator of claim 1, wherein the battery has a black residue rating less than 3.

20. The lead acid battery of claim 19, wherein the black residue rating is less than 2.

21. A lead acid battery comprising the battery separator of claim 1, wherein the battery exhibits
PSoC cycle life testing greater than 10,000 cycles, greater than 20,000 cycles, greater than 30,000 cycles, greater than 40,000 cycles, or greater than 45,000 cycles.

22. A battery separator comprising:
a porous membrane; and a
surfactant that comprises a nonionic surfactant on at least one side of the porous membrane, wherein the the non-ionic surfactant level is from 1 g/m² to 5 g/m².

23. The battery separator of claim 22, wherein the non-ionic surfactant level is from 2 g/m² to 4 g/m².

24. The battery separator of claim 23, wherein the non-ionic surfactant level is from 3 g/m² to 4 g/m².

25. A lead acid battery comprising the separator of claim 22, wherein the battery exhibits at least one of the following longer discharge life, less water loss, improved charge acceptance, longer partial state of charge cycle life, and longer life compared to a battery comprising a separator with a higher non-ionic surfactant level.

26. A battery separator comprising:
a porous membrane; and a
surfactant, wherein the surfactant of the surfactant consists of a compound having one of the following structures: 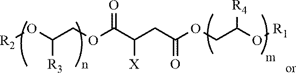 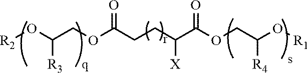
where n is an integer from 0 to 10, m is an integer from 0 to 10, n and m are the same or different, q is an integer from 0 to 10, r is an integer from 0 to 10, s is an integer from 0 to 10, and q, r, and s are the same or different, R1 is H, a C1 to C10 linear or branched, saturated or unsaturated alkyl group, a C1 to C10 fatty alcohol, a C1 to C10 alcohol, or an aromatic group, and R2 is H, a C1 to C10 linear or branched, saturated or unsaturated alkyl group, a C1 to C10 linear or branched, saturated or unsaturated fatty alcohol, a C1 to C10 linear or branched, saturated or unsaturated alcohol, or an aromatic group, n and m are the same or different, R1 and R2 are the same or different, R3 is hydrogen or methyl or a C1 to C5 alkyl group, R4 is hydrogen or methyl or a C1 to C5 alkyl group R3 and R4 are the same or different, and X is a negatively charged groups such as SO3—, COO—, PO4-2, and the like; and also a positively charged counter-ion.

27. The battery separator of claim 26, wherein R3 and R4 are the same and are both hydrogen in either structure; wherein R3 and R4 are the same and both methyl groups in either structure; wherein X is SO3—; wherein X is PO4-2; wherein m and n are each an integer from 1 to 5; wherein m and n are each an integer from 6 to 10; wherein m is an integer from 1 to 5; wherein n is an integer from 1 to 5; wherein m is an integer from 6 to 10; wherein n is an integer from 6 to 10; wherein q, r, and s are each an integer from 1 to 5; wherein q, r, and s are each an integer from 6 to 10; wherein q is an integer from 1 to 5; wherein r is an integer from 1 to 5; wherein s is an integer from 1 to 5; wherein q is an integer from 6 to 10; wherein r is an integer from 6 to 10; or wherein s is an integer from 6 to 10.

28-45. (canceled)
